(19) United States
(12) Patent Application Publication
(10) Pub. No.: US 2009/0305331 A1
(43) Pub. Date: Dec. 10, 2009

(54) Kits and methods for determining colon cleanliness

(75) Inventors: Shomron Ben-Horin, Ramat-Efal (IL); Tidhar Shalon, Tel-Aviv (IL); Guy Kotlizky, Kfar-Shemaryahu (IL); Amir Barzilay, Kochav Yair (IL)

Correspondence Address: MARTIN D. MOYNIHAN d/b/a PRTSI, INC., P.O. BOX 16446, ARLINGTON, VA, 22215, US

(73) Assignee: SVIP 1 LLC, Palo Alto, CA (US)

(21) Appl. No.: 12/461,454

(22) Filed: Aug. 12, 2009

Related U.S. Application Data
(63) Continuation-in-part of application No. PCT/IL08/00203, filed on Feb. 14, 2008.
(60) Provisional application No. 60/902,086, filed on Feb. 20, 2007.

(51) Int. Cl. C12Q 1/02

(52) U.S. Cl. 435/29

(57) ABSTRACT

Method and kits for determining a cleanliness level of a colon of a subject are provided. The methods and kits of the present invention determine a parameter associated with colon cleanliness in a sample obtained from the subject and associate the parameter with the cleanliness level of the colon of the subject.

### RELATED APPLICATIONS

This application is a continuation-in-part (CIP) of PCT Patent Application No. PCT/IL2008/000203 filed on Feb. 14, 2008, which claims the benefit of priority of U.S. Provisional Patent Application No. 60/902,086 filed on Feb. 20, 2007, the contents of which are incorporated herein by reference.

### FIELD AND BACKGROUND OF THE INVENTION

The present invention relates to kits and methods for determining colon cleanliness, and in particular to kits and methods suitable for use with a bowel preparation regimen in order to determine the effect of the bowel preparation on colon cleanliness.

Colonoscopy is the current standard for evaluation of the colon. Other procedures such a capsule colonoscopy (see Schoofs et al, Endoscopy 2006; 38 (10) 971-977 and Eliakim et al, Endoscopy 2006; 38 (10) 963-970) as well as self-propelled colonoscopy (Vucelic et al, Gastroenterology 2006; 130 (3) 672-677), are also emerging as effective alternatives to traditional colonoscopy. In addition, non-invasive radiological imaging of the large bowel by CT colonography, MR virtual colonography, have shown diagnostic utility in detection and screening for colonic disorders. Diagnostic accuracy and therapeutic safety of a colonic examination by any of these methods depend on the quality of bowel preparation as achieved via colon cleansing products and procedures.

Although currently marketed colon cleansing products (also termed purgatives, laxatives, cathartics or lavage solutions) are efficient in cleansing the bowel from fecal matter, over 20% of subjects scheduled for colonoscopy fail to adequately prepare for the procedure (Ness et al. Predictors of inadequate preparation for colonoscopy. Am J Gastroenterol 2001; 96(6):1797-802). Colon cleanliness is generally reported using a scale of 1 to 4 (poor, fair, good, excellent) for each section of the colon, according to Minimal Standard Terminology of digestive endoscopy (Delvaux et. Al Endoscopy 2000; 32: 162-188). Inadequate bowel preparation for colonoscopy can result in missed lesions, cancelled, repeated or more frequent procedures, increased procedural time, and a potential increase in complication rates.

High volume polyethylene glycol (PEG) and low volume sodium phosphate solutions are the most commonly used purgatives for colonoscopy bowel preparations. Polyethylene glycol has some disadvantages, as it requires oral intake of 3 to 4 liters of a very unpalatable salty tasting solution over a short period of time. This large volume causes nausea, vomiting, abdominal fullness, and discomfort, resulting in at least 5-15% of the patients being unable to comply with the entire volume of preparation (Grundel et al. Improvements in mechanical bowel preparation for elective colorectal surgery. Dis Colon Rectum 1997, 40:1348-1352).

Sodium phosphate is an electrolyte solution that provides quality bowel cleansing while avoiding the need to ingest large amounts of solution. Mechanical bowel preparation using this sodium phosphate involves two 45 ml doses, administered a few hours apart, each followed by ingestion of approximately 1 liter of water. The use of sodium phosphate is not recommended for patients with chronic renal failure, and should be used with caution in patients with advanced hepatic cirrhosis, significant heart failure, and ascites.

Due to poor compliance related mostly to palatability, attempts have been made to produce and market improved purgatives. Such attempts have failed since they did not make the cleansing regimen more acceptable to the patient, nor did they produce satisfactory results with respect to colon cleanliness.

Although palatability of currently marketed purgatives influences cleansing quality, less than 20 percent of patients with an inadequate colonic preparation reported a failure to adequately follow preparation instructions, demonstrating that additional patient-specific factors play a role in colon cleansing failures (Ness et al. ibid).

Thus, the present inventors believe that adequate preparation for a colonic procedure is hampered by the lack of indication as to colon cleanliness, prior to the actual colonic examination. As such, the inventors propose that the quality of colonic preparation can be substantially improved if the subject or health care professional is provided with feedback as to the degree of colon cleanliness during and following a cleansing regimen, which would enable to supplement the preparation regimen and/or alter the timing of colonic examination. The present inventors believe that failure to adequately prepare for a colonic procedure in some patients is also dependent upon inter-individual differences in colonic features, such as the motility characteristics of the colon, the degree of mucosal leakage, the amount and consistency of stool at the commencement of the preparation and so forth. Thus, the quality of colonic preparation would be substantially improved if the preparation regimen could be individualized, and preemptively modified in those patients characterized by the methods and kit of the present invention. In addition, knowing that the clinician might require the patient to submit the results of a colonic cleanliness test before starting the colonoscopy examination may increase the compliance of the patient with the colonic preparation procedure.

### SUMMARY OF THE INVENTION

According to one aspect of the present invention there is provided a method of determining a cleanliness level of a colon of a subject comprising determining a parameter associated with colon cleanliness in a sample obtained from the subject and associating said parameter with the cleanliness level of the colon of the subject.

According to further features in preferred embodiments of the invention described below, the subject is scheduled for a colon procedure.

According to still further features in the described preferred embodiments the colon procedure is colonoscopy.

According to still further features in the described preferred embodiments the colon procedure is computed tomographic (CT) colonography

According to still further features in the described preferred embodiments the colon procedure is magnetic resonance (MR) colonography

According to still further features in the described preferred embodiments the colon procedure is colonic capsule endoscopy

According to still further features in the described preferred embodiments determination is effected prior to and/or following colon cleansing.

According to still further features in the described preferred embodiments the sample is selected from the group consisting of a colonic wash sample, a urine sample, a blood sample and a breath sample.

According to still further features in the described preferred embodiments the parameter is the weight of the total colonic wash output, or its volume.

According to still further features in the described preferred embodiments the parameter is a chemical oxygen demand level of the colonic wash sample.

According to still further features in the described preferred embodiments the parameter is a bilirubin or urobilinogen or urobilin/urobilirubin level of the colonic wash sample, the urine sample, or the blood sample.

According to still further features in the described preferred embodiments the parameter is a methane or hydrogen gas level of the breath sample.

According to still further features in the described preferred embodiments the parameter is a bacterial load of the colonic wash sample.

According to still further features in the described preferred embodiments the parameter is a level of particulate matter in the colonic wash sample.

According to still further features in the described preferred embodiments the method further comprises administering a prebiotic to the subject prior to the determining the parameter.

According to another aspect of the present invention there is provided a kit for determining a cleanliness level of a colon of a subject comprising a device for determining a parameter associated with colon cleanliness in a sample obtained from the subject and for associating the parameter with the cleanliness level of the colon of the subject.

According to still further features in the described preferred embodiments the sample is selected from the group consisting of a colonic wash sample, a urine sample, a blood sample and a breath sample.

According to still further features in the described preferred embodiments the device is capable of determining a chemical oxygen demand level of the colonic wash sample.

According to still further features in the described preferred embodiments the device is designed for collecting the colonic wash sample.

According to still further features in the described preferred embodiments the device includes an indicator for determining the chemical oxygen demand level of the colonic wash sample.

According to still further features in the described preferred embodiments the indicator is potassium dichromate.

According to still further features in the described preferred embodiments the device is capable of determining a bilirubin or urobilinogen or urobilin/urobilirubin level of the colonic wash sample, the urine sample, or the blood sample.

According to still further features in the described preferred embodiments the device is capable of determining a methane or hydrogen gas level of the breath sample.

According to still further features in the described preferred embodiments the device is capable of determining a bacterial load of the colonic wash sample.

According to still further features in the described preferred embodiments the device is capable of determining a level of particulate matter in the colonic wash sample.

According to still further features in the described preferred embodiments the kit further comprises guidelines for correlating the measured parameter with the cleanliness level of the colon of the subject.

According to still further features in the described preferred embodiments the kit further comprises a purgative preparation.

According to yet another aspect of the present invention there is provided a kit for quantifying particulate matter in colonic effluent comprising an effluent capturing device and a particulate matter quantifying device.

According to yet another aspect of the present invention there is provided a kit for quantifying the weight and/or volume of the total colonic effluent comprising an effluent capturing device and a weight/volume quantifying device

According to still further features in the described preferred embodiments the effluent capturing device is a funnel having at least one container.

According to yet another embodiment of the present invention, there is provided a kit comprising of indigestible markers to be ingested by the subject prior to or during the ingestion of purgatives

According to still further features of the present invention, the method and device are capable of detecting the number of markers retained in the gastrointestinal tract, and the number expelled therefrom.

According to still further features in the described preferred embodiments the kit further comprises guidelines for correlating the number of markers expelled by the subject, or retained within the abdomen, with the cleanliness level of the colon of the subject.

The present invention successfully addresses the shortcomings of the presently known configurations by providing a kit and method useful for determining the degree of colon cleanliness in a subject. The present kit and method provide the subject with an indication of the quality of bowel preparation and thus minimizes the likelihood that the subject arrive unprepared for a colon procedure and be turned away or be subjected to an examination under suboptimal conditions.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 illustrates one embodiment of a system for determining colon cleanliness constructed in accordance with the teachings of the present invention.

FIG. 2 illustrates a device for collecting a colon wash sample and for determining the quantity of particulate matter in such a sample.

FIGS. 3a-b illustrate the markers used in a study correlating ingestion of markers and colon cleanliness.

FIG. 4 is a colonoscopy image showing the markers in the colon.

FIG. 5 is an X-ray image showing markers in the rectosigma region.

FIGS. 6a-b are graphs showing the correlation between markers present above the pelvis and colon cleanliness (FIG. 6a, r=0.45, p=0.006), and between expelled markers and colon cleanliness (FIG. 6b, r=0.31, p=0.04).

### DESCRIPTION OF THE PREFERRED EMBODIMENTS

The present invention is of kits and methods which can be used to assess the degree of colon cleanliness in a subject. Specifically, the present invention can be used to directly or indirectly estimate the amount of fecal matter present in colon of subject and thus determine the bowel preparation state of the subject.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Outcome of colonoscopy is directly dependent on the cleanliness of the colon. Thus, thorough cleansing of the bowel is necessary before a colonoscopy. Although colon cleansing preparations are effective at cleansing the colon from fecal debris, effective use thereof is limited by the lack of indication as to the degree of colon cleanliness during and following use of such preparations and prior to actual colonic visual examination. This is especially true in cases where colonoscopy is performed using an ingestible capsule. In such cases, colon cleanliness is imperative in order to achieve optimal results [see, Schoofs et al, Endoscopy 2006; 38 (10) 971-977 and Eliakim et al, Endoscopy 2006; 38 (10) 963-970].

While reducing the present invention to practice, the present inventors have devised systems, kits and methods which can be utilized to determine the degree of colon cleanliness during and following colon cleansing and prior to colonoscopy or other colonic procedures. Such systems, kits and methods can be utilized by an individual undergoing colon cleansing procedure or by medical staff prior to commencement of a colon procedure, thereby substantially reducing problems resulting from poor colonic cleansing, such as missed lesions, cancelled procedures, increased procedural time, and a potential increase in complication rates [Rex D K, et al, Am J Gastroenterol 2002; 97:1696-700].

Thus, according to one aspect of the present invention, there is provided a method of determining a cleanliness level of a colon of a subject. As used herein, the term subject refers to an animal, preferably a mammal such as a human which is scheduled for a colon procedure such as colonoscopy, CT colonography, MR colonography, colonic capsule endoscopy (e.g. Pillcam™) or colon surgery (e.g. colon resection, polyp removal).

As used herein, the phrase cleanliness level, refers to the amount of solid material (e.g. feces, fibers etc), and/or liquid (e.g. liquid feces) and/or to the amount of bacteria present in the colon. A clean colon is substantially free of solid matter, liquid feculent material, and has a low bacterial load (less than 10⁴ cfu in a milliliter of a colonic wash effluent).

The method is effected by determining a parameter associated with colon cleanliness in a sample obtained from the subject and correlating the parameter with the cleanliness level of the colon of the subject. As is further described hereinunder, such a parameter can be derived from an exogenous or an endogenous marker.

As is mentioned hereinabove, such determination is performed prior to a colon procedure in order to ascertain the level of cleanliness of the colon and the readiness of the subject to undergo a colon procedure.

Thus, the method of the present invention is preferably practiced during and following bowel preparation and thus serves as an indication to the subject as to the effect of the cleansing regimen.

Several types of samples which are obtained from the subject can be used by the method of the present invention, including, but not limited to, a colonic effluent (e.g. wash sample), a fecal sample, an anal swab, a urine sample, a blood sample or a breath sample.

As is mentioned above, parameters present in such samples can be correlated with a degree of colon cleanliness. The following description provides several examples of parameters which can be used to determine colon cleanliness according to the method of the present invention.

Ingested Markers

It is known that passage of indigestible markers through the alimentary tract correlates with intestinal motility, and thus radio-opaque markers are often employed in evaluation of constipation disorders (see for example Barloon T J et al, Am Fam Physician 1997; 56:512-520). Results shown in a recent publication (Eliakim et al Ibid) suggest that colon motility and thus movement through a colon can be correlated with colon cleanliness by showing that additional purgative doses are associated with increased migration of an ingested colonoscopy capsule. Thus, the inventors contend that ingestion of exogenous markers and testing of passage of such markers through the colon can be correlated with colon cleanliness.

Such markers can be naturally occurring molecules (fibers), dye molecules incorporated into a purgative, a component of the purgative (e.g. PEG) or synthetic particles which can be consumed prior to, or during colon cleansing. The quantity or presence/absence of such markers can be assessed in the colon or colon effluent ejected into the toilet bowl to thereby determine colon cleanliness. For example, in the case of dye molecules or PEG, concentration of the dye color or PEG in an effluent can serve as an indication of cleanliness.

Synthetic markers can include a predetermined number of indigestible, differently tagged or sized/shaped marker particles. Such particles can be, for example, spherical in shape (solid or hollow), roughly 1-15 mm in diameter, and possess a specific color and a specific gravity (e.g. less than 1.0 gr/ml). Such particles can be ingested by an individual at certain time points prior to colonic examination, corresponding to the timing of drinking the colonic purgative. Different markers can indicate each of the respective drinking events in a bowel preparation treatment and allow the purgative effect to be tracked in real time or by the endpoint of expulsion through the GI system of each purgative serving.

Passage of the markers and their evacuation in an effluent sample is determined by direct observation of their appearance in the feces. For example, specific gravity of the markers, can make them float in the toilet water and facilitate their detection and quantification.

Alternatively, one can employ a counter (which can be positioned within the toilet bowl or adjacent to it, or attached to the body of the subject) which can quantify the particles based on a physical property of the particles.

FIG. 1 illustrates one embodiment of a system for determining colon cleanliness which is referred to herein as system 10.

System 10 includes a plurality of ingestible particles 12 and a detector device 14 for counting such particle in a colon wash sample ejected into the toilet.

Particles 12 can be endowed with a property such as radioactivity, magnetism, conductance, fluorescence, sonic signature, RFID tags, or the like and detector device 14 configured for quantifying passed particles based on the detected property. One example of a particle counting device can be a magnetic collector which can collect magnetizable particles (for example iron particles) from the toilet water. Such a collector can include a voltage gate which is closed by the collected magnetizable particles. In such a counting device, the current would be directly proportional to the quantity of magnetizable particles collected and the readout can be a visual display such as a light.

Particles 12 can also include a miniature RF antenna 16 in which case detector device 14 can be RF detector which is mounted on or near the subject or on the toilet seat. Evacuation of each particle 12 will be detected through RF link 19 by detector device 14, and the number of particles 12 evacuated will be displayed on an attached visual screen 18.

The ratio of passed marker particles to un-passed ones can serve as qualitative measurement of gut cleanliness. Moreover, retention of particles in the bowel can be also objectively determined in a non-invasive manner immediately prior to procedure, by having the doctor or the nurse apply a sensitive detector (e.g. metal detector) over the patients' abdomen, to indicate the presence and the colonic location of particles having metallic properties. For example, in capsule colonoscopy, a receiver unit is placed around the abdomen of the patient to communicate with and receive data and images from the capsule. Such a receiver unit can contain a detector tuned to the properties of the ingested particles, which by way of example can be coated iron particles detected from outside the body by the receiver unit in a manner similar to a metal detector. Once a lower threshold of particles is detected by the receiver unit, it can signal to the patient to swallow the imaging capsule. The receiver unit can track the presence, quantity and motion of the particles in the colon or any other location in the user's body in real time and estimate the time required to achieve cleanliness or the probability of achieving cleanliness based on the dynamic nature of these measurements. The receiver unit can be designed to detect any other parameter characteristic of the ingested particles.

Detection of excreted markers can also be effected using a color producing mechanism provided in the marker particles. For example, color producing marker particles which produce a distinct color when in contact with toilet water (which can be pretreated to provide the color activation conditions; e.g. by changing their pH) can be ingested several hours following ingestion of the purgative. Ejection of the particles into toilet water along with the colonic effluent will be identified via appearance of a distinct color in toilet water, the color can be restricted to the particles or it can diffuse therefrom to provide a more pronounced indication.

The presence of retained markers in the gut can be used as an indication of colon cleanliness. Alternatively, retention of markers can be determined by other methods, such as abdominal plain x-ray film, or a RF based system or an electromagnetic system, wherein the signal generated by the markers is detected by an appropriate detector, e.g. a magnetometer, that is externally applied over the abdomen.

Localization of the markers within the GI tract can be used to predict cleanliness. For example, markers detected in the right colon may indicate poor preparation, while markers in the left colon may indicate adequate preparation.

By using such detectable markers, one can tailor fit the preparation regimen to each individual patient. Currently, the pre-determined set volume of purgative to be consumed is a major cause of patient discomfort and non-compliance. Depending on individual physiologic parameters, colons of some individuals may be well prepared following ingestion of smaller amounts of a purgative, whereas others may need a larger volume. The use of detectable markers enables accurate adjustment of the preparation regimen according to individual needs. Specifically, the rate and/or quantity of markers passed by an individual can be used to determine the preparation regimen. If a certain pre-determined number of markers are detected, indicating an effectively cleaned colon, the patient can be instructed to discontinue drinking the purgative. Alternatively, the patient may be instructed to drink an additional volume of the purgative and/or ingest additional cathartics, if a cutoff number of markers had not been passed. In such cases, the colonoscopy examination, swallowing of an endoscopy capsule or the performance of MR or CT colonography may be postponed until a clean colon is obtained in order to optimize colonic visualization and ensure a successful procedure.

The Example section which follows describes a study which clearly correlates presence of markers in the colon with colon cleanliness.

Chemical Oxygen Demand

Chemical oxygen demand (COD) is used as a measure of oxygen requirement of a sample that is susceptible to oxidation by strong chemical oxidant. The dichromate reflux method is preferred over procedures using other oxidants (e.g. potassium permanganate) because of its superior oxidizing ability, applicability to a wide variety of samples and ease of manipulation. Oxidation of most organic compounds is 95-100% of the theoretical value.

COD is very sensitive to presence of even minute amounts of organic matter and thus can be used to detect presence of fecal matter, fibers and bacteria, as well as bacterial components in a colonic wash sample.

Reagents used in COD can be applied directly to a colonic wash sample or to filtered solids thereof, in which case they can be embedded in a filtering device. Use of filtered solids is preferred since some colonic purgatives include PEG as well as ions which interfere with the results, although one can discount the contribution of PEG and ions to the results via a simple conversion chart since the concentration of these components in colonic purgatives is known.

Additional information regarding the COD test and the reagents is provided by Clair et al. (2003) in Chemistry for Environmental Engineering and Science, 5th edition, New York: McGraw-Hill.

A typical COD test of a 1 ml filtered sample of a colonic wash following a proper colon cleansing should provide a COD index of no more than 10 mg/L. A COD index which is significantly higher than that is an indication of poor bowel preparation.

Bilirubin/Urobilinogen/Urobilirubin

Colonic wash, blood or urine samples can be used to determine the level of bilirubin or urobilirubin or urobilinogen and correlate such level with colon cleanliness.

Bilirubin is a yellow breakdown product of normal heme catabolism. Its levels are elevated in certain diseases and it is responsible for the yellow color of bruises and the brown color of feces. Bilirubin reduction in the colon by resident bacteria leads to a product called urobilinogen, some of which is reabsorbed and excreted in urine [Midtvedt and Gustafsson Acta Pathol Microbiol Scand 1981 April; 89(2):57-60]. Urobilinogen, in turn, is further metabolized by intestinal bacteria to urobilin/urobilirubin which impart the brown color to the feces.

Studies have shown that animals with sterilized GI tracts display sub detectable levels of urine and fecal urobilinogen (Gustafsson and Lanke J Exp Med. 1960 Dec. 1; 112:975-81). Thus, urobilinogen is a strong indicator of bacterial load and thus of colon cleanliness.

Indeed several studies have shown that urobilinogen can be used as a fecal pollution indicator (Miyabara et al. Bull Environ Contam Toxicol. 1994 July; 53(1):77-84 and Miyabara Environ Pollut. 1994; 84(2):111-5).

Thus, by using simple chromogenic indicators of urobilinogen and/or urobilin/urobilirubin, the method of the present invention can be used to determine colon cleanliness level from a colonic wash or urine sample. It is expected that effective colonic cleansing regimens would produce near sub-detectable levels of urobilirubin in urine samples and low levels of urobilinogen in colonic wash samples.

Numerous approaches for urobilinogen quantification are known in the art. One commonly used test for urobilinogen is based on a diazotisation reaction of 4-Methoxybenzene diazoniurn salt and urobilinogen in an acid medium.

Test strips for urobilinogen quantification in urine samples are also well known [Fetter and Rupe Am J Med. Technol. 1981 September; 47(9):729-35]. In such tests the color changes from pink to brown-red in samples having a concentration of at least 0.1 mg/dl.

Bilirubin levels can also be used to assess the degree of colon cleanliness. Since bilirubin is typically present in fecal matter, detection of bilirubin in filter-captured or wipe-captured particulate matter can provide an indication of fecal residue left in the colon. In this sense, bilirubin serves as surrogate for fecal matter which is hard to detect with naked eyes. Bilirubin detection is based on the coupling of bilirubin with 2.4-dichlorobenzene diazonium salt in an acid medium. The color changes from light tan to pinkish-purple. The test has a sensitivity threshold of 0.5 mg/dl. In this embodiment, the use could be provided with a special wipe that he or she uses at the end of a bowel movement much like toilet paper, and then this wipe is dropped in container containing 2.4-dichlorobenzene diazonium salt in an acid medium and the color of the wipe is examined for determining cleanliness level of the colon.

Bacterial Load/Bacterial Typing

Depending on dietary intake, feces of a human subject includes a bacterial fraction of 15-50% w/w [Kurasawa et al. Journal of the American College of Nutrition, Vol. 19, No. 4, 426-433 (2000)].

The flora of the large intestine (colon) is qualitatively similar to that found in feces. Colon bacteria can reach levels of 10¹¹/ml in feces. The predominant bacterial species in feces are anaerobic bacteria: Bacteroides species, such as Bacteroides vulgatus, clostridial species such as members of the genera Faecalibacterium and Eubacterium and lactic acid bacteria of the genus Bifidobacterium (Bifidobacterium bifidum). In feces, these organisms may outnumber E. coli by 1,000:1 to 10,000:1.

Although most bacteria loosely associate with the colon mucosa, some bacteria adhere specifically to the gastrointestinal epithelial surfaces. Gram-positive bacteria, such as the streptococci and lactobacilli, are thought to adhere to the gastrointestinal epithelium using polysaccharide capsules or wall lipoteichoic acids to attach to specific receptors on the epithelial cells. Likewise, Gram-negative bacteria such as the enterobactria may attach by means of specific fimbriae on the bacterial cell which bind to glycoproteins on the epithelial cell surface.

Since most colon bacteria are either found in stool or loosely associated with the mucous membrane, colon cleansing depletes colon bacteria populations and thus over time, a gradual decline in bacterial load should be observed. Thus, by examining colon wash samples captured from colonic effluent (i.e. liquid ejected from the rectum), an end point of colon cleansing could be determined. Although bacterial load and diversity in the colon varies from one individual to the next, it can be assumed that a bacterial load of 10-10⁴ per ml of a colon wash sample represents a colon which is substantially free of particulate matter.

Several approaches can be used to measure bacterial load including bacteria-specific dyes, ligands or substrates.

Bacterial dyes such as the Gram stain (crystal violet) can be used to identify and quantify bacteria such as lactobacilli and bifidobacterium. Additional bacterial dyes which can be used include the fluorescent stain acridine orange that can be combined with antibodies to increase sensitivity.

Ligands such as lectins, bacteria-specific antibodies and the like can also be used to identify and quantify bacteria in a wash sample.

Lectins are receptor proteins of non-immune origin that specifically interact with sugar molecules (carbohydrates) without modifying them. These proteins recognize and bind specifically to monosaccharides and are classified by which sugar they recognize. Most lectins recognize either N-acetylneuraminic acid, N-Acetylglucosamine, N-Acetylgalactosamine, galactose, mannose, or fucose.

Lectins which specifically bind bacterial polysaccharides are well known in the art. Gram-negative bacteria display lectin receptors on capsules, cytoplasmic membranes, outer membranes, as lipopolysaccharides, lipooligosaccharides, peptidoglycan, surface array glycoproteins while Gram-positive bacteria display lectin receptors on capsules and surface arrays, as group-specific polysaccharides, lipoteichoic acids, peptidoglycans, teichoic acids, and teichuronic acids. For a detailed description of lectin use in microbiology see Calderon et al. (Lectins, Biology, Biochemistry, Clinical Biochemistry, Volume 12, including Proceedings from the 17th International Lectin Meeting in Wurzburg, 1997, edited by Edilbert van Driessche, Sonia Beeckmans and Thorkild C. Bog-Hansen).

Lectins can be directly used to aggregate bacteria present in the wash sample, in which case aggregated bacteria can be directly visualized, or a chromogenic moiety can be added to the lectin (e.g. peroxidase) in order to facilitate bacterial visualization and quantification via a color producing reaction.

Bacteria-specific antibodies are well known in the art and widely used in a variety of diagnostic assays. Test strips for a variety of bacteria species are commercially available (Orgenics; http://orgenics.com). Such strips can be fabricated for use with the present invention by producing antibodies specific to protein and carbohydrate constituents of colon bacteria such as Lactobacilli or bifidobacterium.

Numerous bacteria-specific substrates which detect the presence of bacteria or bacteria-derived enzymes are known in the art. For example, the chromogenic substrate methylene blue is biochemically reduced by intestinal bacteria to its colorless form leucomethylene blue (Prosst et al. European Surgical Research 2005; 37:246-249). Additional substrates which may be used by the method of the present invention are described by Manafi et al [Microbiol Mol Biol Rev. 1991 September; 55(3): 335-348].

Since some bacterial species are more closely associated with the mucous lining of the colon and thus may be released last during colon cleansing, it stands to reason that such bacteria could serve as an indicator for colon cleanliness. For example, the level of Bacteroides vulgatus in a colonic wash sample should increase towards the end of cleansing. Thus, measuring the level of this bacteria before and after bowel preparation should give an indication as to colon cleanliness. Since one would expect bifidobacteria to be washed out more easily than Bacteroides vulgatus, a ratio of these two bacteria types should also change during cleansing, whereas in an unclean colon the levels of bifidobacteria would be higher that those of Bacteroides vulgatus in a wash sample, in a clean colon, Bacteroides vulgatus levels would be much higher than those of bifidobacteria

Sample colonic effluent to be assayed can be collected using a wipe (much like a piece of toilet paper), on a filter paper, in a container or from the toilet bowl water. A calorimetric assay can indicate the quantitative or qualitative presence or absence of total bacterial load or of a specific form of bacteria.

Particulate Matter

Particulate matter in the colon is largely composed of bacteria, dietary fibers, moisture and fats. The amount of particulate matter released with the colon wash is directly related to colon cleanliness. Thus, quantification of particles in a colon wash sample can be used to determine colon cleanliness.

Several approaches can be used for such quantification. A collection tube filled with colonic effluent can be assayed for turbidity using simple light attenuation spectrometry or the like. Alternatively, a filter paper funnel can be used to trap particles and provide an assessment of particle density, or a funnel with graduated-stepped sides and a flow through aperture or a funnel with wash capturing inlets can be also used. Particulate matter can sediment in a sample collection tube over time and the height of the sediment column can be an indicator of cleanliness, with a quantitative scale, such as a correlation to, for example the 1-4 scale of the Minimal Standard Terminology in digestive endoscopy, see Delvaux et. Al Endoscopy 2000; 32: 162-188) 1 indicates no more than small bits of adherent feces, 2 indicates small amounts of feces or dark fluid, but not enough to interfere with the examination, 3 indicates enough fecal or dark fluid to preclude a completely reliable examination and 4 indicates large amount of fecal residue. Alternatively, the indicator can provide a qualitative color scale for the patient to read (for example green indicates a clean colon and red indicates a dirty colon).

Since fecal particles include bacteria (as much as 50% by weight), identification and quantification of such particles can be enhanced by using bacterial stains such as those described above. Bacterial staining will enhance particle visualization and provide a better measure of particle count. One can use the suspended solids (SS) index and methodology to assess particle concentration.

Fecal tags can also be employed to enhance detection of fecal particles in the colon wash sample. Dietary fecal tag preparations (e.g. barium containing contrast agents) are well known in the art, see for example, Weishaupt et al. Lancet 1999; 354:835-836.

### Total Colonic Effluent Weight/Volume

Because the commonly used purgatives are isosmolar passing the colon without absorption, and because the volume and weight of normal daily feces output in humans are known, the total volume of colonic effluent output during preparation, and its weight, may be employed to indicate the degree of cleanliness achieved.

H₂ and Methane

Breath hydrogen and methane are specific end products of colonic fermentation. Hydrogen and methane are end products of fermentation that are absorbed into the portal bloodstream and excreted via expired air. Levels of hydrogen in expired breath have been shown to correlate very well with concentrations produced in the large intestine (Wilkens et al. Cancer Epidemiology, Biomarkers & Prevention Vol. 3, 149-153, March 1994).

Since the formation of hydrogen and methane is unique to anaerobic bacteria present in the gut, and since no mammalian cell is known to produce these gases, methane and hydrogen levels in expired air provide an excellent measure of the amount of methane/hydrogen producing bacteria found in the colon.

Thus, quantification of methane and hydrogen from a breath sample could serve as a marker for colon cleanliness, the less gas expired, the cleaner the colon.

Such quantification can be effected using a breath analyzer calibrated for identifying and quantifying hydrogen and methane. Since such analyzers are typically bulky and expensive spectroscopy devices, the subject undergoing colon cleansing can collect breath samples at several time points and have these samples analyzed in a medical facility. Alternatively, a simple chemical or electrical based analyzer can be used by the subject during and following cleansing.

Since breath hydrogen and methane levels are low and thus difficult to quantify using home use analyzers, production of such gasses in a home setting can be increased by providing methanogens and hydrogen producing bacteria with a substrate such as a prebiotic (e.g. non digestible corn starch). The increased methane and hydrogen production would be easier to detect and quantify prior to colon cleansing, providing the user with baseline of methane and hydrogen levels to which levels following cleansing can be compared.

Mucin

Epithelial surfaces in the human gastrointestinal tract are covered by a layer of mucus, which prevents most microorganisms reaching and persisting on the mucosal surface. The mucus gel mainly consists of water (95%) and glycoproteins such as mucin (1 to 10%), which are responsible for its viscosity and gel-forming properties, as well as electrolytes, proteins, antibodies, and nucleic acids.

Mucins are chemically and structurally diverse molecules, but they invariably contain large quantities of galactose and hexosamines with lesser amounts of fucose.

It has been suggested that certain Bifidobacterium and Ruminococcus strains are numerically dominant populations degrading mucin oligosaccharides in the human colon [Hoskins et al. J Clin Invest. 1985 March; 75(3):944-53]. The breakdown products of mucin (mucin derived sugars) are utilized as a source of energy by mucin degrading bacteria as well as other gut bacteria.

It has been shown that mucin-derived sugars increase in feces of germ-free rats and that fecal excretion of mucin-derived sugars increased almost threefold in germfree rats [Cabotaje et al. Appl Environ Microbiol. 1994 April; 60(4): 1302-1307].

Thus, a degree of colon cleanliness can be assessed from the level of mucin and mucin components (e.g. sugars) present in a colon wash sample. Antibodies directed against mucin and mucin components are available commercially and can be used to assess the levels of such components in a colon wash sample.

Any of the methods described herein can be practiced during and following colon cleansing. An absolute value representing cleanliness can be determined and used to mark an end point of washing. In such a case, a subject simply tests a parameter during and following cleansing and determines the level of cleanliness and the readiness for colon procedure. Alternatively, one can also test the parameter at the beginning of the cleansing regimen and use that value as a reference point for the degree of cleanliness. This is especially useful in cases where a parameter varies from one individual to a degree where an absolute value of cleanliness would be less accurate.

The above described methods can be practiced using dedicated kits for testing colon cleanliness.

Thus according to another aspect of the present invention there is provided a kit for determining a cleanliness level of a colon of a subject. The kit includes a device for determining a parameter associated with colon cleanliness in a sample obtained from the subject and enable assessment of the cleanliness level of the colon of the subject.

The kit can be designed to measure and optionally quantify a parameter using approaches described hereinabove with respect to the present method. Such a parameter can be a biological parameter or, for example, the number of indigestible markers passed by the individual.

For example, in the case of urobilinogen or bilirubin, the device of the present kit can include test strips as described above or test vials with chromogenic reactants. Such test strips or vials can be used with a colonic wash sample capturing device (e.g. vial or filter) or in any other manner described in this patent application.

To determine particulate matter concentration, the present device can be a filter or funnel as described above. The present device can also be a special purpose toilet paper or a Wet-Wipe™ like sheet that the subject uses at the end of a bowel movement, where a change of color or other noticeable signal provides the indication of bacterial load and thus colon cleanliness.

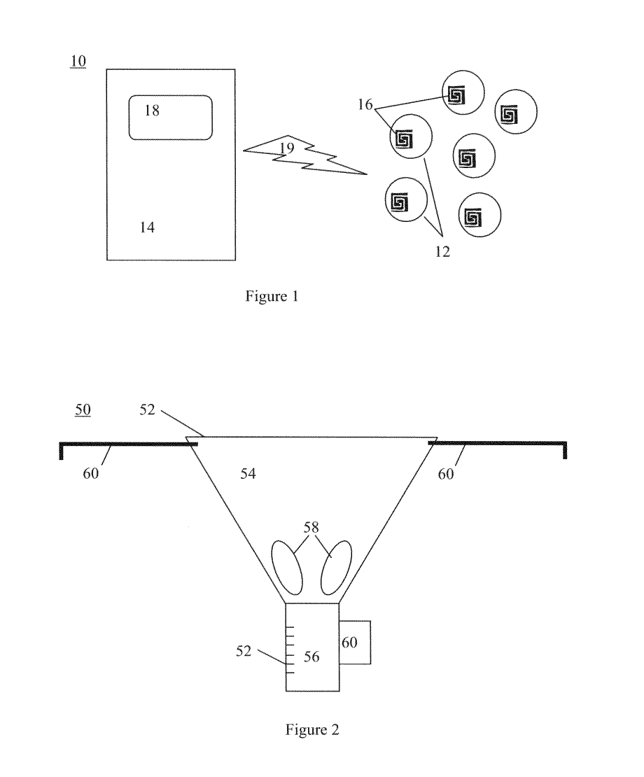

One preferred configuration of a particulate matter capturing/measuring kit employs a colon effluent capturing device and a particulate matter quantifying device. One embodiment of such a kit is illustrated in FIG. 2.

Kit 50 includes a device 52 which includes a funnel 54 and one or more fluid capturing container 56 disposed at the bottom of funnel 54. Side holes 58 prevent overflow of funnel 54. Funnel 54 is shaped and sized for use in capturing a colonic wash effluent of a subject undergoing a bowel preparation. As such, kit 50 can also include elements 60 for attaching funnel 54 to the subject or the toilet seat.

In use, funnel 54 directs the flow of the colonic wash effluent from the subject to the toilet bowl and a sample of the effluent flowing through funnel 54 is captured by container 56.

Following use, kit 50 is retrieved by the subject and containers 56 can be detached and/or sealed. Kit 50 is then positioned upright for several minutes to an hour allowing particulate matter in the colon wash sample captured by containers 56 to settle.

The kit can also include an index 52 for determining the amount of particulate matter in the captured sample. Such an index can provide the subject with an indication of the amount of particulate matter in the effluent which can then be associated with colon cleanliness, via, for example, a lookup table provided by the kit. Alternatively, such an index can directly represent colon cleanliness levels.

In order to facilitate visualization of sedimented particles, containers 56 can also include a bacterial substrate, or a bacterial ligand as described above.

If used with ingested particles, container 56 can be equipped with detector 60 for detection of these particles as described elsewhere in this patent application.

Thus, the kit enables the subject to directly and rapidly assess colon cleanliness and determine if the bowel preparation was successful. It will be appreciated that in cases where the results are indicative of poor bowel preparation, the subject can simply wait for the colon to continue emptying, repeat the colon cleansing regimen or supplement it (by, for example, consuming another bottle of purgative) and retest for colon cleanliness prior to the scheduled colon procedure. This ensures subject readiness for the exam and spares the subject from having to schedule an additional colon procedure due to poor results or reschedule the procedure due to lack of ample preparation. In the case of a capsule endoscopy, the colon cleanliness test can save a subject or the insurance company the cost of a wasted capsule.

The kit may be presented in a pack or dispenser device which may contain one or more unit forms containing the device. The pack may, for example, comprise metal paper or plastic materials. The pack or device may be accompanied by instructions for use in determining colon cleanliness. The pack or device may also be accompanied by a notice in a form prescribed by a governmental agency regulating the manufacture, use, or sale of diagnostic devices, which notice is reflective of approval by the agency of the form of the device for human or veterinary use. Such notice, for example, may include labeling approved by the U.S. Food and Drug Administration for diagnostic devices or of an approved product insert.

### EXAMPLE

Reference is now made to the following example, which together with the above descriptions, illustrates the invention in a non limiting fashion.

### Ingestible Markers as Predictors of Colon Cleanliness

A study was undertaken in order to investigate if there is a correlation between passage of ingestible markers and the degree of colon cleanliness for colonoscopy and in order to determine parameters such as optimal time of markers ingestion with relation to lavage ingestion, and cut-off number of passed markers.

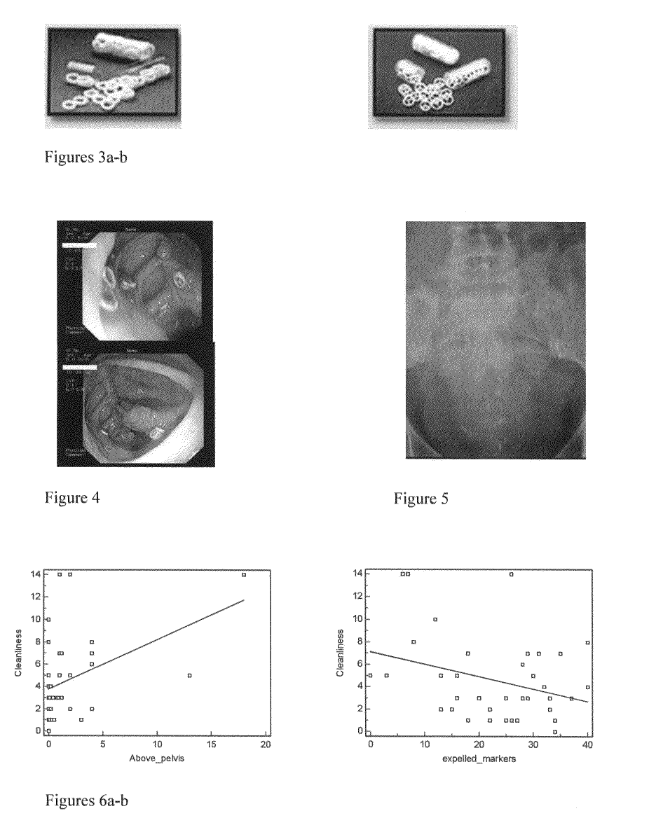

Forty three hospitalized colonoscopy candidates between the ages of 18 and 75 were used in this study. Each candidate ingested 2 capsules, one including O-shaped (FIG. 3a) radio-opaque markers (Sitzmark markers, Konsyl Pharmaceuticals, Md., USA) and the other including tripod-shaped (FIG. 3b) radio-opaque markers. Each candidate then consumed 4 sachets of Fortrans (Pharma Swiss) with 4 liter water. No other laxative, purgatives, enemas or pro-kinetic agents were consumed during the colonic preparation. Laxatives used routinely were withheld 48 hours prior to colonoscopy.

Results

Of the 43 patients recruited 5 patients were excluded due to technical reasons and as such, 38 patients were available for analysis. None of the patients suffered from adverse events related to the ingested markers, and colonoscopy was not affected by the presence of markers in the bowel (FIG. 4).

Low to modest correlation (r=0.31) was achieved for expelled markers (i.e. not seen at X-ray at all), likely resulting from accumulation of markers seen at recto-sigma (FIG. 5 for an exemplary patient). Better correlation was observed for markers above pelvis, i.e. above the recto-sigma, (r=0.45, p=0.006) strongly supporting marker movement along the colon as predictor of cleanliness (FIG. 4)

The results of the present study conclusively show that markers can be safely administered to patients undergoing preparation for colonoscopy, and that the movement of markers along the colon after ingestion of lavage can be correlated with colon cleanliness and readiness for colonoscopy.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A kit for determining a cleanliness level of a colon of a subject comprising a device for determining a parameter associated with colon cleanliness in a sample obtained from the subject and for associating said parameter with the cleanliness level of the colon of the subject.

2. The kit of claim 1, wherein said sample is selected from the group consisting of a colonic wash sample, a urine sample, a blood sample and a breath sample.

3. The kit of claim 1, wherein said device is designed for collecting said colonic wash sample.

4. The kit of claim 1, wherein said device is capable of determining a bilirubin or urobilinogen or urobilirubin level of said colonic wash sample, said urine sample, or said blood sample.

5. The kit of claim 1, wherein said device is capable of determining a methane or hydrogen gas level of said breath sample.

6. The kit of claim 1, wherein said device is capable of determining a bacterial load of said colonic wash sample.

7. The kit of claim 1, further comprising a plurality of indigestible markers, wherein said device for determining said parameter associated with colon cleanliness is designed for determining a quantity of said markers in a sample obtained from the subject or retained within the body of the subject.

8. A method of determining a cleanliness level of a colon of a subject comprising determining a parameter associated with colon cleanliness in a sample obtained from the subject and associating said parameter with the cleanliness level of the colon of the subject.

9. The method of claim 8, wherein said subject is scheduled for a colon procedure.

10. The method of claim 9, wherein said colon procedure is colonoscopy, CT colonography, capsule endoscopy, MR colonography.

11. The method of claim 8, wherein said determining is effected prior to, during, and/or following colon cleansing.

12. The method of claim 8, wherein said sample is selected from the group consisting of a colonic wash sample, a urine sample, a blood sample and a breath sample.

13. The method of claim 12, wherein said parameter is a bilirubin or urobilirubinogen level of said colonic wash sample, said urine sample, or said blood sample.

14. The method of claim 12, wherein said parameter is a methane or hydrogen gas level of said breath sample.

15. The method of claim 12, wherein said parameter is a bacterial load of said colonic wash sample.

16. The method of claim 8, further comprising administering indigestible markers to the subject prior to said determining said parameter.

17. The method of claim 8, wherein said parameter is a quantity of said indigestible markers present in a colonic wash sample and/or retained and present in the patient digestive tract.
